(19) United States
(12) Patent Application Publication
(10) Pub. No.: US 2013/0018302 A1
(43) Pub. Date: Jan. 17, 2013

(54) SINGLE-USE CARDIOVASCULAR DEVICE FOR MEDICAL-SURGICAL OPERATION

(71) Applicants: Sayed Nour, Chaville (FR); Pierre Chastanier, Paris (FR)

(72) Inventors: Sayed Nour, Chaville (FR); Pierre Chastanier, Paris (FR)

(21) Appl. No.: 13/574,607

(22) PCT Filed: Jan. 19, 2011
(86) PCT No.: PCT/EP2011/050703

§ 371 Date: Oct. 5, 2012

(30) Foreign Application Priority Data
Jan. 22, 2010 (FR) 1050428

(51) Int. Cl. A61M 25/10; A61M 1/00

(52) U.S. Cl. 604/30; 604/99.01

(57) ABSTRACT

The invention relates to a single-use device, intended to be used in surgery each time a vascular approach by cannulation or catheterization (extracorporeal circulation, anesthesia, emergency, intensive care) is found to be necessary, in particular in heart surgery or interventional cardiology. This new device essentially includes a body (5), a sealing system consisting of two inflatable disks (4), a control connector for inflating and deflating the disk (4), a tubular unit (6) and a flexible guide (1).

This invention relates to a single-use device, intended to be used in surgery each time a vascular approach by cannulation or catheterization (extracorporeal circulation, anesthesia, emergency, intensive care) is found to be necessary, in particular in heart surgery or interventional cardiology. This new device enables more effective and much safer penetration of the blood vessels and/or cardiac cavities than the devices currently used.

Extracorporeal circulation (ECC) is a well-known medical technique, used in cardiac assistance as a total temporary replacement of the heart and lungs in operating units, or only as a partial replacement in cardiac catheterization and intensive care units, both in pediatrics and in adults.

In general, an ECC console is comprised of an arterial head pump (centrifugal or peristaltic) and four other peristaltic pumps intended for the cardiotomy suction and cardiac chambers and for administration of the cardioplegia. It is all completed by a backup pump as well as biocompatible equipment, tubings, arterial and venous cannulae, venous reservoir, oxygenator, arterial filter, and so on.

The relay between ECC and the patient's body requires specific equipment (tubings, cannulae and catheters, in particular) that are adapted to the morphology and the hemodynamic criteria of each part of the heart and vessels involved:

a) the arterial line of the ECC is connected to an aortic or femoral cannula;

b) the venous blood arrives through one or two cannulae, introduced into the superior and inferior vena cava or the right atrium;

c) a left ventricle discharge cannula is introduced at the apex of the heart, the left atrium or the pulmonary artery;

d) a catheter intended for injection of the cardioplegia is slid into the aortic root upstream of the aortic cannula, or through the right atrium into the coronary sinus or directly into the coronary ostia;

e) supplementary catheters enable hemodynamic measurements to be obtained: systemic and pulmonary blood pressure line, venous lines including a central line, a pressure line into the left atrium and so on.

Surgical operations affecting the heart and vessels use delicate techniques, specifically in pediatric heart surgery, with high risks of hemorrhage and trauma.

The aortic cannulation, for example, which must, once in place, be perfectly safe, requires “purse”-string suture points to be produced, kept on pull members before piercing the aorta by means of a bistoury, and the cannula to be pushed in very quickly so as to prevent a cataclysmic hemorrhage.

This cannula will then be connected, with precaution, to the arterial line of the ECC in order to prevent any risk of gas embolism in the circuit. The same procedure will then be repeated to position the cardioplegia catheter or the cannula.

An aortic clamp will be positioned downstream of the cardioplegia catheter when the ECC is started (the heart will stop after injection of the cardioplegia).

These invasive methods reserved for experienced surgeons can sometimes lead to considerable vascular complications. In newborns with a very short aortic root, or in elderly people having a so-called “porcelain” aorta, the risks of arterial dissection, thromboembolic syndrome, or fractures of the atheromatous plaque may appear.

To overcome these technical problems, the inventors propose a more effective solution that must respond step-by-step to each problem presented by the vascular cannulation. It consists of combining, in a single device, all of the tools enabling a more effective, faster, more secure and less costly cardiovascular approach to be obtained.

The single-use medical vascular catheterization or cannulation device of the invention can be used as:

a) an aortic cannula,

b) a cardiac cannula,

c) a vascular catheter,

d) a drainage cavity tubing.

It includes:

an attachment system, which will replace the purse-string sutures and their pull members;

a system for closing off the blood flow, which will replace the metal aortic clamp that still causes vascular trauma on the endothelial part of the vessel and that can cause breakage of the atheromatous plaque in elderly subjects;

a guided perfusion system, which will replace the cardioplegia cannula, and will separately direct the blood and cardioplegia perfusions precisely and effectively.

With the different systems that it includes, the device of this invention enables complex surgical procedures normally requiring the intervention of an experienced team, to be performed by a single operator, in an entirely safe manner.

An additional advantage of the invention is that the surgical space concerned, such as the periaortic space in the case of an operation targeting the aortic trunk, is less encumbered and enables a better approach to the heart, which therefore has free space around it.

The subject matter of the invention is a cardiovascular device for a surgical intervention enabling a single operator to penetrate the blood vessels and/or cardiac cavities. It consists of a proximal part intended to penetrate said blood vessels cardiac cavities, an intermediate part consisting of the body of said device and a distal part capable of housing at least one catheter and/or connection system.

In a first embodiment, the device of the present invention is characterized in that it includes:

a body 5 having a longitudinal axis XX′ and enabling lines, catheters, vascular tubings and connectors necessary for said surgical operation to be housed and guided,

a sealing system consisting of two inflatable disks 4, which extend substantially perpendicularly to the longitudinal axis XX′ of the body 5, axially separated from one another by several millimeters, with a proximal disk 4a intended to be positioned on the interior wall of a vascular opening or a cardiac cavity, and a distal disk 4b intended to be positioned on the exterior wall of a vascular opening or a cardiac cavity. The two inflated disks ensure the tightness by compression of the vascular wall, with the position of said two disks on the longitudinal axis of said device being adjustable by the operator so as to modify the vascular penetration depth and the two disks being housed in the body of the device so as to avoid any enlargement of the incision during ablation of said device,

a control connector for inflating and deflating said inflatable disks 11 constituting the sealing system, housed in the body 5 of the device,

a tubular unit 6 attached to the exterior wall of the body of the device, containing the rod of said control connector, with the rod being equipped with an anti-return valve system 8,

a flexible guide 1 mobile with respect to the interior of the body 5 of the device, of which the proximal part has a point located near the proximal part of the body of the device and which enables an incision to be made in a vessel or in the heart, with the movement of said guide in the body 5 being controllable by its distal part housed in said lateral tubular unit 6.

In another embodiment, the device according to the invention also includes, in its proximal part intended to penetrate the blood vessels or cardiac cavities:

an inflatable occlusive balloon 2, of which the position can be modified by digital guiding of the surgeon or by remote guiding with respect to the longitudinal axis XX′ of the device, with said inflatable occlusive balloon being placed at the proximal end and integrated with the body of the device so as to avoid any enlargement of the incision during ablation of said device, with the inflation and deflation of said inflatable occlusive balloon being controlled by a connector 10 integrated in the exterior wall of the device and which has a rod, equipped with an anti-return valve system 8 located in the lateral tubular unit 6,

at least one lateral hole 3 in the body of the device, axially located between the inflatable occlusive balloon 2 and the two inflatable disks 4 of the sealing system, enabling cardioplegia injections, which said at least one hole 3 being connected to a cardioplegia perfusion system by a connector 9 integrated on the exterior wall of the device and which has a rod, equipped with an anti-return valve system 8 located in the lateral tubular unit 6.

In a particular embodiment, when it is used as a ventricle discharge cannula, as a non-limiting example, the device of this invention is characterized in that the distal disk 4b, of the two inflatable disks constituting the sealing system, is in the form of a suction cup during its inflation so as to adapt to the shape of the end of the heart.

In particular in this case, without it being restrictive, the device according to this invention is characterized in that the proximal disk 4a, of the two inflatable disks constituting the sealing system, has at least one irrigation hole forming an antithrombotic safety system.

In an embodiment derived from the above embodiments, the cardiovascular device according to one of the previous claims may have a pivot elbow 13 enabling the device to be tilted with respect to the internal vascular or cardiac wall 12.

In these different embodiments, the cardiovascular device is used to implement aortic or cardiac cannulations, vascular catheterizations or cavity drainages.

By body 5 of the device, we mean a tube having a longitudinal axis XX′ made of a suitable material in the field of surgery, combining or serving as a support for the different mechanisms, systems and accessories constituting the device according to the present invention. The body 5 may have lengths and diameters that are variable according to the surgical applications of the device according to the invention, in particular according to the cardiac and pericardial morphologies. In addition, the body 5 of the device may have at least one pivot elbow 13 so as to make the vascular catheterization less complicated and reduce the risks of trauma. The pivoting of the device can be produced manually or in a controlled manner, assisted by a pneumatic, electric or other type of system.

In addition, the proximal part of the device of the invention, intended to penetrate the opening of the vessels and/or the cardiac cavities, may have a curvature adapted to the physiology of the vascular and/or cardiac opening concerned, enabling better flow of the blood between the device and the targeted organ.

Concerning the different systems arranged in the external wall of the device, such as balloons or automatic suture systems, they are integrated in the wall so as to avoid any enlargement or tearing of the vascular or cardiac incision during penetration and/or ablation of the device.

By sealing system, we mean a system enabling the vascular or cardiac wall to be closed by compression on its internal and external faces around the incision produced by the device of the invention, with said sealing system being based on the presence of two inflatable disks 4, one described as proximal 4a and the other described as distal 4b with respect to their positions on the longitudinal axis XX′ of the device, with these two inflatable disks being housed in the wall of the device so as to avoid any enlargement or tearing of the vascular or cardiac incision during penetration and/or ablation of the device. The housings capable of receiving said inflatable disks 4 can be in the form of tunnels of which the diameters are less than the diameter of the body 5 of the device. According to this invention, the proximal disk 4a penetrates the vascular or cardiac opening while the distal disk 4b remains outside. By inflatable disks, we mean two disk-shaped balloons of which the surface after inflation is compatible with compression of the vascular wall, with the surface of the disks after inflation and the compression force having to be perfect so as to ensure a seal at the incision without damaging the vascular or cardiac wall, and without requiring pull members or suture points.

The position of these inflatable disks, spaced apart by several millimeters, can be modified on the longitudinal axis of the device so as to adapt it to the thickness of the vascular or cardiac wall concerned, in the location of the incision, and to be capable of modifying the vascular penetration depth of the device. This positioning may be manual, performed directly or indirectly by the operator, or controlled with the assistance of a pneumatic, electrical or other type of system.

In a particular embodiment, when the device of the present invention is used as a ventricle discharge cannula, the distal disk 4b may be in a so-called “suction cup” form during inflation, enabling it to be adapted to a compression of the external wall of the apex of the heart. Concomitantly, the proximal disk 4a may be equipped with an irrigation system so as to prevent the local formation of micro-clots between the inflatable balloon and the vascular wall; in particular, the proximal disk 4a may be equipped with at least one irrigation hole, and preferably a plurality, so as to enable a heparin serum, for example, to flow. This particular embodiment preferably extends to all applications requiring the cannula to be held on the vascular or cardiac wall for a prolonged period.

The inflation and deflation of said disks constituting the sealing system are triggered directly by the operator via a connector 11 housed in the wall of the body of the device, connected to the rod of said connector 11 located in the tubular unit 6 of the device of the present invention.

By inflatable occlusive balloon 2, we mean primarily an aortic obstruction balloon, positioned several millimeters from the proximal end of the device. The position of said inflatable occlusive balloon 2 can be modified by manual guiding of the surgeon or by remote guiding with respect to the longitudinal axis XX′ of the device, preferably in the intraoperative period. Modifying the position of said balloon means tilting said balloon on its axis so as to position it well with respect to the morphology of the arteries or other openings concerned. This tilt refers to the longitudinal axis XX′ of the device according to the present invention; after inflation, as the axis of said balloon is substantially perpendicular to the axis XX′ of the body of the device, this angle may be modified according to the morphology of the arteries so as to ensure complete occlusion of the vessels and cavities concerned. This possibility of intraoperative guiding, by the surgeon's finger, of the inflatable occlusive balloon, enables, unlike the current catheter systems, an obstruction of the blood flow into the ascending aorta, without blocking the circulation in the vessels of the aortic arch which perfuse the brain and upper limbs. Dramatic complications are thus avoided, such as paraplegia due to blockage of the anterior radicular artery of Adamkiewicz with a traditional larger balloon catheter. Said balloon advantageously replaces the traditional mechanical (titanium or carbon) aortic or vascular clamp, which remains a brutal instrument that injures the endothelial wall. A person skilled in the art knows that such a clamp may cut an aorta if it is not securely attached on the operating field, in a closely monitored place, avoiding any accidental attachment even though this operating field is particularly encumbered when traditional methods and instruments are used.

The inflation and deflation of said inflatable occlusive balloon are triggered via a connector 10 housed in the wall of the body of the device, connected to the rod of said connector 10 located in the tubular unit 6 of said device, directly by the operator.

By lateral opening or hole, we mean at least one, and preferably two orifices 3 in the wall of the device, enabling cardioplegia injections. These openings are located between the inflatable occlusive balloon 2 and the disks 4 of the sealing system, ideally directed toward the coronary ostia. These lateral holes 3 replace the traditional system of cardioplegia injection by separate catheter and avoid the risks and complications associated with such vascular complications, encumbering of the aorta, and so on.

This or these lateral hole(s) 3 are connected to a cardioplegia perfusion system by a connector 9 integrated on the external wall of the device and which has a rod equipped with an anti-return valve system 8, located in the lateral tubular unit 6.

By flexible guide 1 or guide, we mean a flexible rod that is relatively mobile inside the body of the device, according to the longitudinal axis XX′ and of which the proximal part is a point that enables an incision to be made in the wall of a vessel and/or a cardiac cavity. The movement of said guide is controlled manually or by remote guiding by the surgeon, by its distal part housed in the lateral tubular unit 6. The material of said guide is a material compatible with a surgical practice and may be variable, plastic, metallic, and so on. The material of the point of said guide may be similar to or different from that of the guide. The length and diameter of said guide can be adapted according to the use of the device according to this invention and according to the shape of said device, which is related to the morphology of the vessel addressed. The shape of the point of said guide is also adapted according to the incision that is to be made; it may take the shape of a more or less thick blade, a conical point or a more or less thick needle, and so on. In a particular embodiment, the means for incising of the device of this invention may be a laser beam.

This flexible guide 1 makes it possible to do without the traditional steps encountered when making an incision, usually performed by two operators, one incising with a bistoury blade and the other taking care of the blood flow so as to prevent any risk of hemorrhage.

The control of the introduction of said flexible guide 1 in an artery may be manual, performed directly or indirectly by the operator, or controlled with the assistance of a pneumatic, electric or other type of system. A simple pressure enables the point of said guide 1 to be introduced into the artery, quickly followed by the body of the device, avoiding the risk of vascular tears. Once the pressure has been released by the operator, the cutting point of the guide 1 goes into the body of said device, enabling the body of the device to be introduced into the vascular cavity in an entirely safe manner.

By tubular unit 6, we mean a compartment attached to the exterior wall of the body of the device according to this invention and containing the rods of the lateral opening control connectors, the sealing system, the inflatable occlusive balloon and also the distal part of the flexible guide, as well as the anti-return valves of each of the connectors.

The figures below, provided by way of an illustrative and non-limiting example, will make it easier to understand how the device of the present invention works:

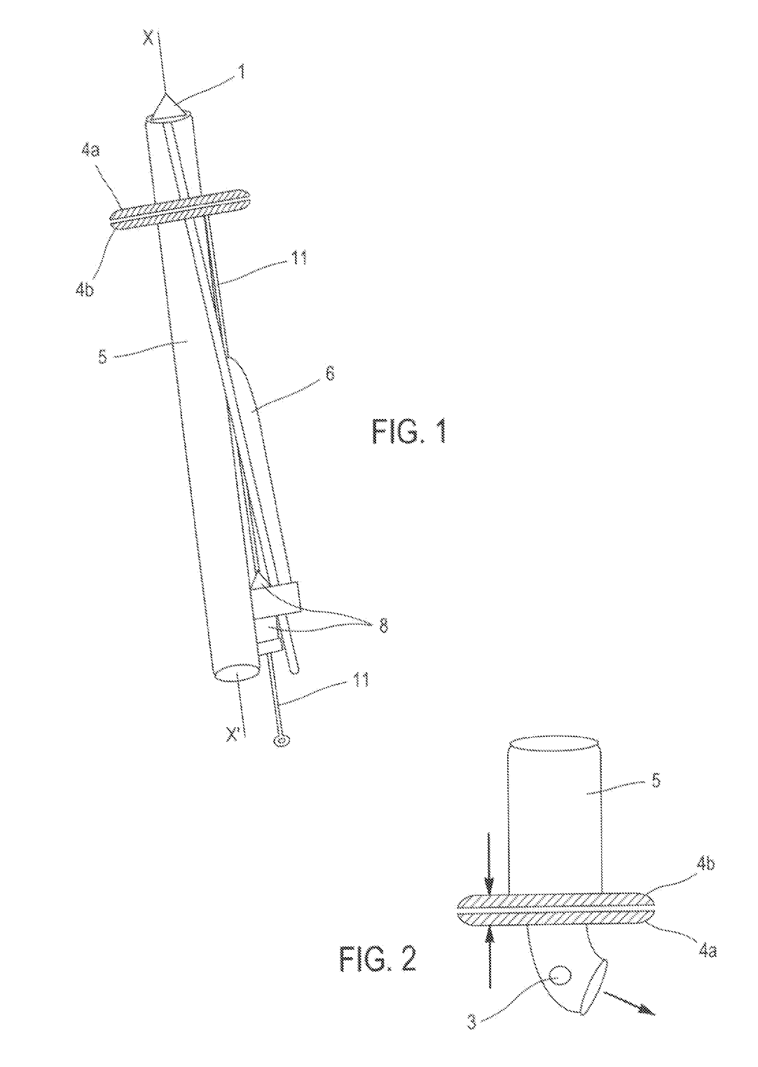

FIG. 1: longitudinal frontal cross-section showing the device according to the invention in its non-aortic cannula or catheter version.

FIG. 2: longitudinal profile cross-section showing the proximal end of the device according to the invention in its non-aortic cannula or catheter version.

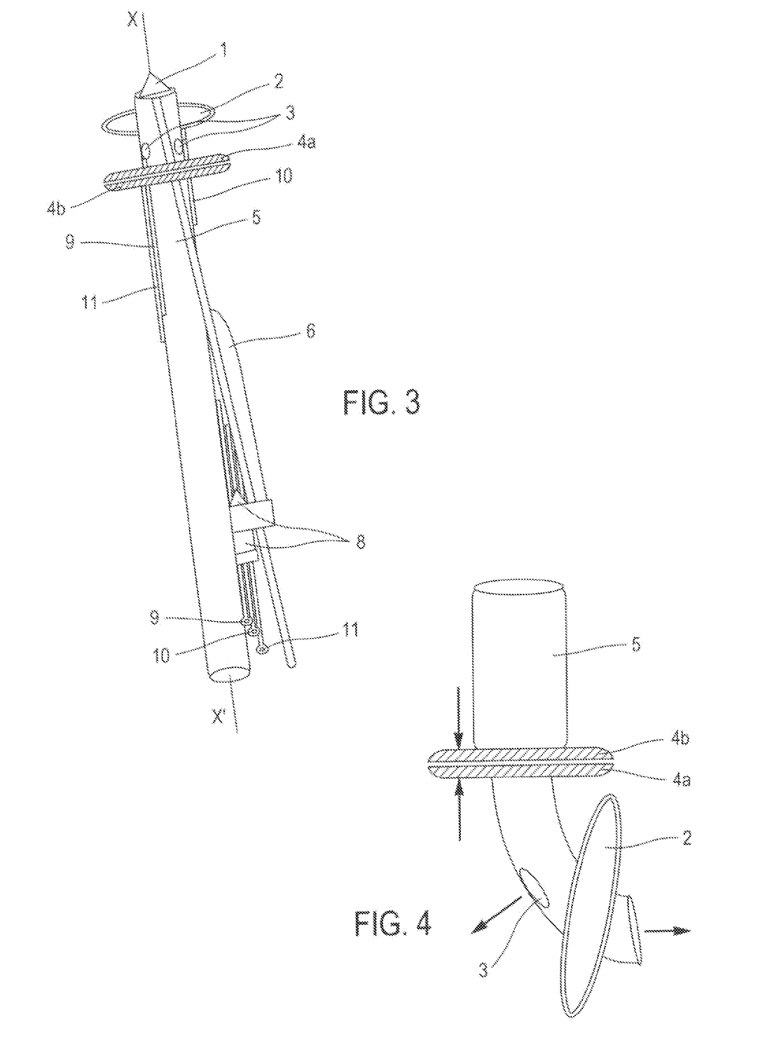

FIG. 3: longitudinal frontal cross-section showing the device according to the invention in its aortic cannula version.

FIG. 4: longitudinal profile cross-section showing the proximal end of the device according to the invention in its non-aortic cannula or catheter version.

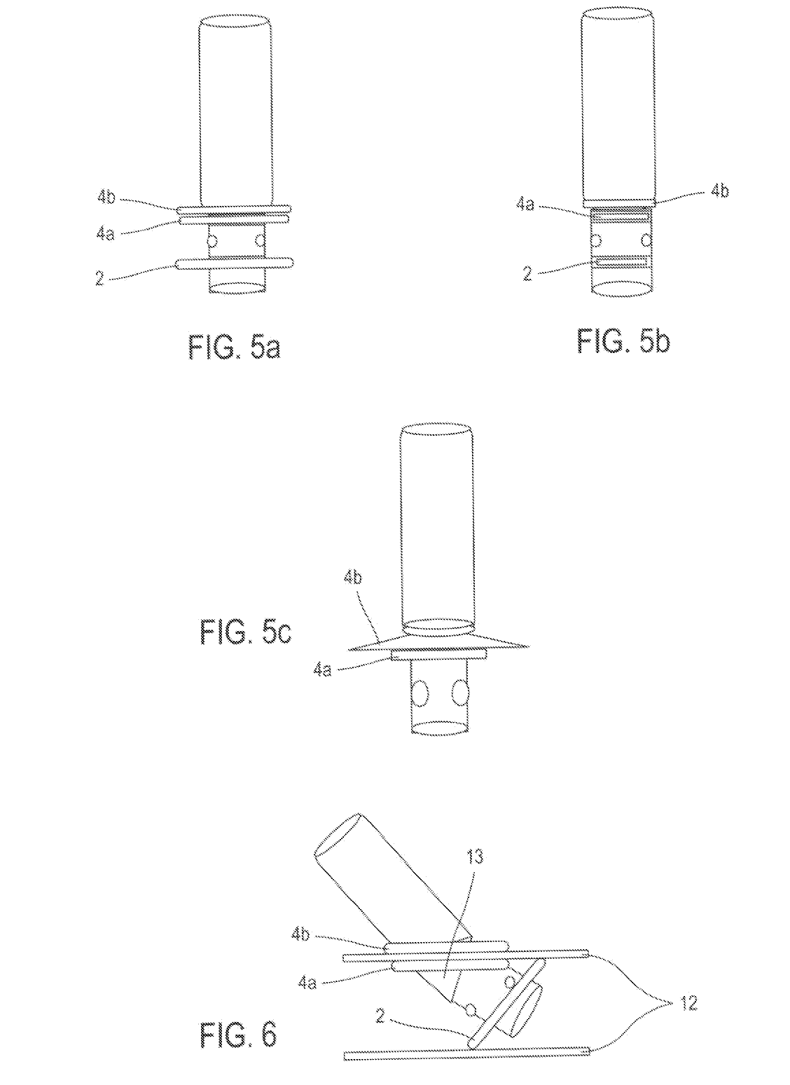

FIG. 5: longitudinal frontal cross-section showing the inflation (FIG. 5a) and the deflation (FIG. 5b) of the inflatable occlusive balloon and the disks constituting the sealing system; FIG. 5c: longitudinal frontal cross-section showing the inflation in the form of a suction cup of the distal disk 4b.

FIG. 6: longitudinal frontal cross-section showing the device according to the invention equipped with a pivot elbow.

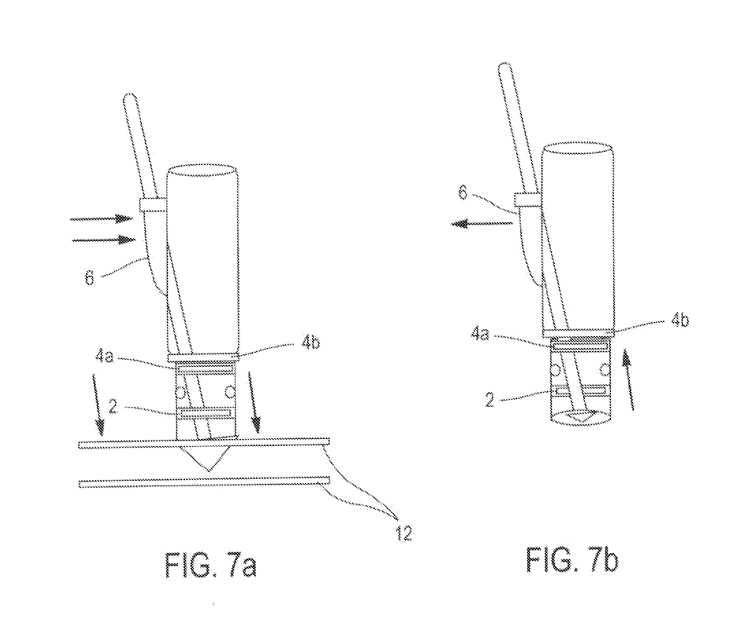

FIG. 7: longitudinal frontal cross-section showing the incision of a vessel by the metallic guide of the device of the invention (FIG. 7a) and the retraction of same (FIG. 7b).

1. Cardiovascular device for a surgical intervention enabling a single operator to penetrate the blood vessels and/or cardiac cavities, consisting of a proximal part intended to penetrate said blood vessels and cardiac cavities, an intermediate part consisting of the body of said device and a distal part capable of housing at least one catheter and/or connection system, characterized in that it includes:
a body (5) having a longitudinal axis XX′ and enabling lines, catheters, vascular tubings and connectors necessary for said surgical operation to be housed and guided,
a sealing system consisting of two inflatable disks (4), which extend substantially perpendicularly to the longitudinal axis XX′ of the body (5), axially separated from one another by several millimeters, with a proximal disk (4a) intended to be positioned on the interior wall of a vascular opening or a cardiac cavity, and a distal disk (4b) intended to be positioned on the exterior wall of a vascular opening or a cardiac cavity, the two inflated disks ensuring the tightness by compression of the vascular wall, with the position of said two disks on the longitudinal axis of said device being adjustable by the operator so as to modify the vascular penetration depth and the two disks being housed in the body of the device so as to avoid any enlargement of the incision during ablation of said device,
a control connector for inflating and deflating said inflatable disks (11) constituting the sealing system, housed in the body (5) of the device,
a tubular unit (6) attached to the exterior wall of the body of the device, containing the rod of said control connector, with the rod being equipped with an anti-return valve system (8),
a flexible guide (1) mobile with respect to the interior of the body (5) of the device, of which the proximal part has a point located near the proximal part of the body of the device and which enables a vascular or cardiac incision, with the movement of said guide in the body (5) being controllable by its distal part housed in said lateral tubular unit (6).

2. Cardiovascular device according to claim 1, characterized in that it also includes, in its proximal part intended to penetrate the blood vessels or cardiac cavities:
an inflatable occlusive balloon (2), of which the position can be modified by digital guiding of the surgeon or by remote guiding with respect to the longitudinal axis XX′ of the device, with said inflatable occlusive balloon being placed at the proximal end and integrated with the body of the device so as to avoid any enlargement of the incision during ablation of said device, with the inflation and deflation of said inflatable occlusive balloon being controlled by a connector (10) integrated in the exterior wall of the device and which has a rod, equipped with an anti-return valve system (8) located in the lateral tubular unit (6),
at least one lateral hole (3) in the body of the device, axially located between the inflatable occlusive balloon (2) and the two inflatable disks (4) of the sealing system, enabling cardioplegia injections, which said at least one hole (3) being connected to a cardioplegia perfusion system by a connector (9) integrated on the exterior wall of the device and which has a rod, equipped with an anti-return valve system (8) located in the lateral tubular unit (6).

3. Cardiovascular device according to claim 2, characterized in that the distal disk (4b), of the two inflatable disks constituting the sealing system, is in the form of a suction cup during its inflation so as to adapt to the shape of the end of the heart.

4. Cardiovascular device according to claim 3, characterized in that the proximal disk (4a), of the two inflatable disks constituting the sealing system, has at least one irrigation hole forming an antithrombotic safety system.

5. Cardiovascular device according to claim 4, characterized in that it has a pivot elbow (13) enabling the device to be tilted with respect to the internal vascular or cardiac wall (12).

6. Cardiovascular device according to claim 2, characterized in that the proximal disk (4a), of the two inflatable disks constituting the sealing system, has at least one irrigation hole forming an antithrombotic safety system.

7. Cardiovascular device according to claim 6, characterized in that it has a pivot elbow (13) enabling the device to be tilted with respect to the internal vascular or cardiac wall (12).

8. Cardiovascular device according to claim 2, characterized in that it has a pivot elbow (13) enabling the device to be tilted with respect to the internal vascular or cardiac wall (12).

9. Cardiovascular device according to claim 3, characterized in that it has a pivot elbow (13) enabling the device to be tilted with respect to the internal vascular or cardiac wall (12).

10. Cardiovascular device according to claim 1, characterized in that the distal disk (4b), of the two inflatable disks constituting the sealing system, is in the form of a suction cup during its inflation so as to adapt to the shape of the end of the heart.

11. Cardiovascular device according to claim 10, characterized in that the proximal disk (4a), of the two inflatable disks constituting the sealing system, has at least one irrigation hole forming an antithrombotic safety system.

12. Cardiovascular device according to claim 11, characterized in that it has a pivot elbow (13) enabling the device to be tilted with respect to the internal vascular or cardiac wall (12).

13. Cardiovascular device according to claim 1, characterized in that the proximal disk (4a), of the two inflatable disks constituting the sealing system, has at least one irrigation hole forming an antithrombotic safety system.

14. Cardiovascular device according to claim 13, characterized in that it has a pivot elbow (13) enabling the device to be tilted with respect to the internal vascular or cardiac wall (12).

15. Cardiovascular device according to claim 1, characterized in that it has a pivot elbow (13) enabling the device to be tilted with respect to the internal vascular or cardiac wall (12).

16. Cardiovascular device according to one of the preceding claims, wherein the device is configured to implement aortic or cardiac canules.

17. The cardiovascular device according to claim 1, wherein the device is configured to implement vascular catheters.

18. Cardiovascular device according to one of the preceding claims, wherein the device is configured to implement cavitar drainages.
